(19) United States
(12) Patent Application Publication
(10) Pub. No.: US 2023/0140908 A1
(43) Pub. Date: May 11, 2023

(54) METHOD FOR COMPREHENSIVE MANAGEMENT OF A HEALTH CONDITION

(71) Applicants: Jayant Nemchand Lokhande, Cerritos, CA (US); Sonali Jayant Lokhande, Cerritos, CA (US)

(72) Inventors: Jayant Nemchand Lokhande, Cerritos, CA (US); Sonali Jayant Lokhande, Cerritos, CA (US)

(21) Appl. No.: 17/524,532

(22) Filed: Nov. 11, 2021

(51) Int. Cl. G16H 20/90; G16H 50/70; G06Q 30/06; G09B 5/04; G09B 5/06; G09B 5/02

(52) U.S. Cl. CPC G16H 20/90; G16H 50/70; G06Q 30/0631; G09B 5/04; G09B 5/065; G09B 5/02

(57) ABSTRACT

A method for comprehensive management of a principal health condition is described. The method broadly comprises the steps of receiving a principal health condition from the beneficiary, explaining the principal health condition and associated health condition to the beneficiary, formulating healthcare products for the principal health condition, the associated health condition and the root cause factor of the principal health condition based on nine elements of life, recommending the formulated healthcare products to the beneficiary, increasing awareness of the beneficiary regarding the extra corporeal components of the principal health condition, suggesting him/her a plurality of lifestyle modification tips so as to enhance the effectiveness of the healthcare products, and providing the beneficiary with relevant literature regarding the principal health condition.

### FIELD OF THE INVENTION

The present invention relates to the field of healthcare. Particularly, the present invention relates to a method for comprehensive management of a health condition.

### BACKGROUND OF THE INVENTION

The World Health Organization (WHO) defines health as “a state of complete physical, mental, and social well-being and not merely the absence of disease or infirmity”. This clearly indicates that for an individual to be called as “healthy”, other aspects also need to be taken care of apart from the physical infirmity. However, most of the health management, treatments or plans proposed to an individual do not take all the aspects of a health condition (disease/ailment/disorder) into consideration. As a result, the only beneficial effect that can be seen on an individual is in terms of the medicines proposed to him/her. This does not ensure a comprehensive management of a health condition since the other aspects remain unaddressed.

All physiological systems in living organisms work in tandem i.e. there isn't a single physiological system that functions independently or in isolation. In fact, a given physiological function is a net sum of deep and diverse physiological functions network. This is known as Systems Biology in simplest way. Such an integrated physiological network determines the eventual fate and scope of a particular health condition.

Any given health condition may further have an associated health condition in the body. For instance, the trigger of Alzheimer's or Dementia might start in Hepatobiliary System. Further, Alzheimer's is usually closely connected with conditions like Eye Disorders, Insomnia etc. Hence, to manage a health condition like Alzheimer's, it is important to take care of such associated health conditions in order to manage it with maximum effectiveness. It is equally important to address the root cause factor(s) of a particular health condition in order to manage it effectively.

Furthermore, any given health condition has a corporeal (physical) component and an extra corporeal (metaphysical) component. Every healthcare condition is a resultant of imbalance of and/or mixture of organic and non-organic or physical and metaphysical factors (body and consciousness). Both these components play a critical role in the manifestation of a health condition. The primary elements of life that affect the corporeal branch of a particular health condition include Pruthvi, Aakasha, Jal, Vayu, and Tejas whereas the elements which affect the extra corporeal branch of a particular health condition include Mansa, Kaala, Atman and Disha.

Addressing all these complex aspects governing the fate of a particular health condition becomes important in its management. However, till date, there does not exist a health management plan or method which takes care of all these aspects together, in order to address a particular health condition.

Yet another bottleneck in the effective management of a health condition is the lack of awareness in the patients or individuals (end consumers) who intend to manage their health condition, regarding the selection of appropriate healthcare products for the health condition. There are selection options available to such individuals. However, they are clueless about which healthcare products would be the best to manage their health condition in context.

Furthermore, as far as the healthcare products are considered, there are multiple aspects which need to be taken into consideration as well. The quality of healthcare products depends on the active and non-active ingredients used, the formulation process adopted, the price and the utility/therapeutic value. For example, Curcumin requires fats as a media for the best absorption in human body and hence liposomal curcumin is a better choice as compared to its capsule or tablet form. An average individual's education and medical awareness is usually inadequate to understand the above factors while selecting healthcare products. As a result, he/she may end up selecting or opting for a healthcare product which does not really address the health condition in context in the best possible way.

Further in this context, even the store manager of a medicine store is incapable of recommending the best healthcare products due to various limiting factors. Also, most of the healthcare practitioners such as NDs, CAM, Chiropractor, Physiotherapist, Store managers, Wellness and health coaches, and MDs do not usually practice Systems Pharmacology while suggesting or advising healthcare products. Empirically, most of the healthcare product brands are influenced by social, economic, political, cultural, environmental factors and do not keep individual customer value as the central aim. Consequently, individuals looking for good healthcare products for managing their health condition do not get best, knowledgeable and unbiased advice or recommendation for selection of the products.

### PRIOR ART

JP2011006371A describes a system that performs health management by the inquiry information of lifestyle due to the change in the physical condition of the user. The system includes obtaining and storing physical condition information of a user in different databases, a measurement unit that measures the current physical condition of the user, a detection unit, an input unit and an output unit, a analysis unit and a report unit. The health management feedback of the system includes any one or more of dietary, exercise or behavioral information for managing the health condition of the user. However, the system does not include formulation and/or recommendation of healthcare products to the user based on his health condition.

JP2013080570A describes a health management device and a method for user health management using a user's behaviour standard and behaviour information. However, this method fails to take the actual health condition into consideration and relies on behaviour based information provided by the user.

In the current scenario, there is no well integrated and comprehensive health management method which takes all the of the aspects stated herein above into consideration while managing a particular health condition. Hence, there is a need for a comprehensive method for management of a health condition which takes into consideration the complexity of a health condition, addresses multiple aspects of the same and keeps the maximum effective management of a health condition as the central focus.

### OBJECTS OF THE INVENTION

A primary object of the present invention is to provide a method for comprehensive management of a health condition.

Another object of the present invention is to provide a method for comprehensive management of a health condition that addresses both the corporeal component and the extra corporeal component of a health condition.

Yet another object of the present invention is to provide a method for comprehensive management of a health condition that addresses the principal health condition, its associated health condition as well as the root cause factors of the principal health condition.

Yet another object of the present invention is to provide a method for comprehensive management of a health condition that recommends healthcare products to the beneficiary based on a Systems Biology or Systems Pharmacology approach.

Yet another object of the present invention is to provide a method for comprehensive management of a health condition that recommends healthcare products whose active and inactive ingredients are incorporated based on the principal health condition, the associated health condition and the root cause factors of the principal health condition.

Yet another object of the present invention is to provide a method for comprehensive management of a health condition, that recommends healthcare products formulated on the basis of nine elements of life—Pruthvi, Jala, Tejas, Vayu, Akasha, Manas, Kaala, Disha, and Atman.

Yet another object of the present invention is to provide a method for comprehensive management of a health condition that recommends healthcare products in the form of a three product package, addressing the principal health condition, the associated health condition and the root cause factors of the principal health condition respectively.

Yet another object of the present invention is to provide a method for comprehensive management of a health condition that increases the user's awareness regarding the principal health condition and the associated health condition.

Yet another object of the present invention is to provide a method for comprehensive management of a health condition that addresses the extra corporeal component of a health condition by means of non-product intervention such as appropriate education and lifestyle modification suggestions, based on the principles and practices of Ashtanga and Kriya Yoga.

Yet another object of the present invention is to provide a method for comprehensive management of a health condition, wherein the appropriate education and lifestyle modification suggestions provided to the user help enhance the effectiveness of the healthcare products recommended for the principal health condition.

### SUMMARY OF THE INVENTION

The present invention relates to a method for comprehensive management of a health condition. In one aspect, the method of the present invention manages the principal health condition of a beneficiary in a comprehensive manner by taking into consideration and addressing the corporeal component and the extra corporeal component of a principal health condition, the associated health condition of the principal health condition and the root cause factors of the principal health condition, altogether.

In another aspect, the method of the present invention includes formulation of plurality of healthcare products and recommendation of a package of three healthcare products namely a principal healthcare product, a first ancillary healthcare product and a second ancillary healthcare product. These products are formulated such that their active ingredients are incorporated based on the principal health condition, the associated health condition and the root cause factors of the principal health condition.

In yet another aspect, the principal healthcare product is formulated to manage the principal health condition, the first ancillary product is formulated to manage the associated health condition and the second ancillary product is formulated to manage the root cause factors of the principal health condition. In yet another aspect, the healthcare products are formulated on the basis of nine elements of life namely Pruthvi, Jala, Tejas, Vayu, Akasha, Manas, Kaala, Disha, and Atman.

In yet another aspect, the method of the present invention further includes the steps of increasing awareness of the beneficiary regarding the principal health condition and the extra corporeal factors associated with it, by means of education and demonstration in audio or visual or textual format. In yet another aspect, the method of the present invention addresses the extra corporeal component of a health condition by means of non-product intervention such as appropriate education and lifestyle modification tips, based on the principles and practices of Ashtanga and Kriya Yoga.

In yet another aspect, the method of the present invention includes management of a health condition of the beneficiary in a way such that the lifestyle modification tips help enhance the effectiveness of the healthcare products. In yet another aspect, the method includes providing the beneficiary with meritorious research articles and literature regarding the principal health condition.

### BRIEF DESCRIPTION OF DRAWINGS

FIG. 1 illustrates a chart showing different components and aspects of a principal health condition.

FIG. 2 illustrates a flowchart describing the overall method for comprehensive management of a health condition in accordance with the general teachings of the present invention.

### DETAILED DESCRIPTION OF THE INVENTION

Before the present invention is described, it is to be understood that this invention is not limited to methodologies described, as these may vary as per the person skilled in the art. It is also to be understood that the terminology used in the description is for the purpose of describing the particular embodiments only and is not intended to limit the scope of the present invention. Throughout this specification, the word “comprise”, or variations such as “comprises” or “comprising”, will be understood to imply the inclusion of a stated element, integer or step, or group of elements, integers or steps, but not the exclusion of any other element, integer or step, or group of elements, integers or steps. The use of the expression “at least” or “at least one” suggests the use of one or more elements or ingredients or quantities, as the use may be in the embodiment of the invention to achieve one or more of the desired objects or results. Various embodiments of the present invention are described below. It is, however noted that the present invention is not limited to these embodiments, but rather the intention is that modifications those are apparent are also included.

Throughout the specification:

“Beneficiary” refers to the end user or consumer of the present invention, typically being but not limited to a patient;

“Principal health condition” refers to the primary health condition the beneficiary intends to manage;

“Associated health condition” refers to any health condition other than the principal health condition that is related to or associated with or accompanied with the principal health condition;

“Healthcare product” refers to any human-ingestible or topically applied product meant for improvement of the overall health of the beneficiary;

“Corporeal component” refers to any bodily or physical component of a given health condition; and

“Extra corporeal component” refers to any non-bodily or non-organic or meta-material or meta-physical component of a given health condition.

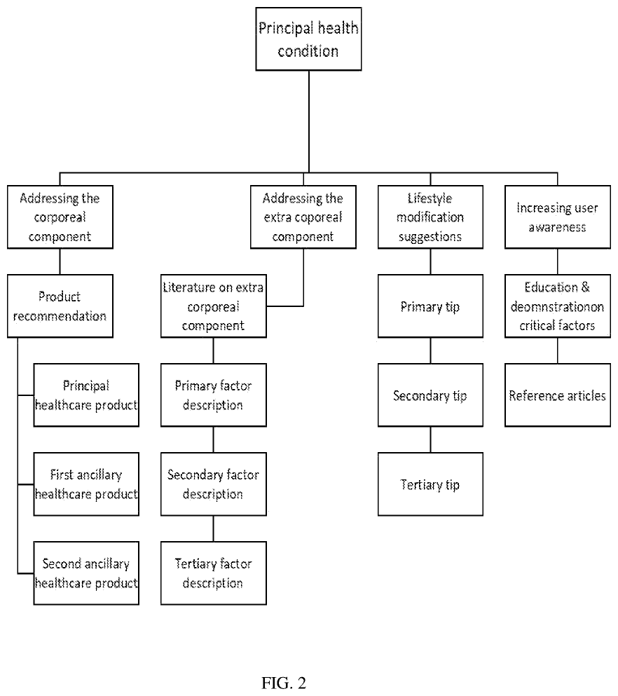

The present invention relates to a method for comprehensive management of a particular health condition of a beneficiary. The method broadly comprises the steps of receiving name of a principal health condition from the beneficiary, explaining the principal health condition and associated health condition to the beneficiary, formulating healthcare products for the principal health condition, the associated health condition and the root cause factor of the principal health condition based on nine elements of life, recommending the formulated healthcare products to the beneficiary, increasing awareness of the beneficiary regarding the extra corporeal components of the principal health condition, suggesting him/her a plurality of lifestyle modification tips so as to enhance the effectiveness of the healthcare products, and providing the beneficiary with relevant literature regarding the principal health condition. The overall method for comprehensive management of health conditions in accordance with the general teachings of the present invention is as illustrated in FIG. 2. Various features of the invention shall be described and become apparent in the description to follow.

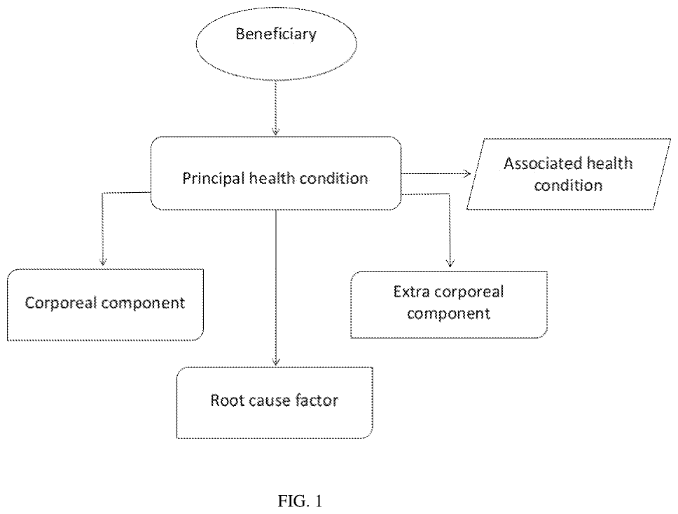

Referring to FIG. 1, the method allows the beneficiary to provide or specify a principal health condition. The principal health condition has a corporeal component and an extra corporeal component. Furthermore, the principal condition may have at least one associated health condition, which also needs to be effectively managed so as to achieve a better result in terms of health condition management.

The method of the present invention is further directed to manage the principal health condition by adopting a System Biology approach. The system biology approach takes into account all physiological processes that are interrelated very closely. For example, heart function is closely related to the kidney function and liver function. The method of the present invention takes both corporeal and extra corporeal components of the principal health condition, the associated health condition as well the root cause factors of the principal health condition into account, and involves formulation of healthcare products to address each of them.

In a preferred embodiment of the present invention, the aforementioned healthcare products are formulated on the basis of nine basic elements of life namely Pruthvi, Jala, Tejas, Vayu, Akasha, Manas, Kaala, Disha and Atman. A brief description of the elements is as given in Table. 1.

TABLE 1

| Sr. No. | Element | Brief description |
|---|---|---|
| 1. | Pruthvi | Energy of Existence |
| 2. | Jala | Energy of Congregation |
| 3. | Agni | Energy of Transformation |
| 4. | Vayu | Energy of Operation |
| 5. | Aakash | Energy of Extension |
| 6. | Manas | Thoughts & Patterns of Thoughts, Intellect, |
| | | Id (Ego), Memory |
| 7. | Kaala | All types and calibrations of Time |
| 8. | Disha | Ten Directions |
| 9. | Atman | Self and Universal Consciousness/Awareness |

Pruthvi (Symbolically Earth): Energy of ‘Existence’

Every Object in the Universe is perceived/felt because it ‘exists’ and has specific dimensions. Energy of Existence brings the ‘Form’ to that object.

Jala (Symbolically Water): Energy of ‘Congregation’

For every Object to ‘exist’, its formative elements must ‘congregate’ in the most optimized way. Otherwise the Object disintegrates apart.

Agni (Symbolically Fire): Energy of ‘Transformation.’

For every Object to manifest all its formative elements must undergo certain ‘transformation’ so that they can compound into each other to form one uniform object. Without such transformation, formation of an object does not occur.

Vayu (Symbolically Air): Energy of ‘Motion’

For every object to manifest, all its formative particles must displace from one place to another in the specific ‘motion & pace’. Without such motion, manifestation of an object does not occur.

Aakash (Symbolically Space): Energy of ‘Extent.’

To manifest an Object, it must have its own ‘extent’. Without such extent, manifestation of an object does not occur.

Manas (Symbolically and in limited sense Mind).

Manas is an entity through which every living object is connected to the universe and vice versa and perform all types of ‘Actions of Life.’

Kaala (Symbolically Time and its calibration).

Kaala is an entity by which every living and nonliving object ‘exist’ in its given ‘form’ for a specified amount of ‘time.’

Disha (Symbolically Direction).

Disha is an Entity by which every manifested object is ‘relatively’ placed to other manifested object, till all relative objects exist.

Atman (Symbolically Atman).

Every living and non-living object has its own embodied consciousness, and that individual embodied consciousness is a sub part of ‘one and universal consciousness’.

The above nine elements can be categorized into five gross elements namely Pruthvi, Jala, Agni, Vayu and Aakash, and four subtle elements namely Manas, Kaala, Disha and Atman. The five gross elements are associated with the corporeal components of a health condition whereas the four subtle elements are associated with the extra corporeal components of the same. The subtle elements indirectly have an influence over the functionality of the gross elements. Further, the gross elements are quantitative in nature while the subtle elements are qualitative in nature. Hence, in the healthcare products formulated on the basis of these nine elements, the proportions of the five gross elements vary depending on the principal health condition whereas the proportions of the four subtle elements stay the same in all the healthcare products. This shall become more apparent and clear by way of an example provided towards the end of the detailed description of the invention.

In a preferred embodiment, the method of the present invention involves recommending the beneficiary a package of three healthcare products based on the above mentioned formulation process. The three healthcare products include a principal healthcare product, a first ancillary healthcare product, and a second ancillary healthcare product. The principal healthcare product is formulated so as to manage the principal health condition, the first ancillary healthcare product is formulated so as to address the associated health condition and the second ancillary healthcare product is formulated so as to address the root cause factors of the principal health condition. In yet another embodiment of the present invention, the package of healthcare products can be bought by the beneficiary in an offline manner or using an online platform.

Further, the recommended healthcare products of the present invention may be in the form of but not limiting to dietary supplements, nutritional supplements, functional foods, phytochemicals, OTC products, active pharmaceutical ingredient, medical foods and topically applied products. It is to be noted that the method of the present invention does not offer any pharmaceutical/regulated drugs per se and rather offers healthcare products which address a particular health condition in a comprehensive manner by addressing the associated health condition and root cause factors of the principal health condition simultaneously/parallelly.

In another preferred embodiment of the present invention, the method for comprehensive management of a health condition is directed towards increasing awareness of the beneficiary towards the principal health condition in context. The awareness step includes non-product intervention in the form of educating, describing and demonstrating the beneficiary, various aspects of the principal health condition. The method of the present invention further provides information and describes the extra corporeal causes of the principal health condition to the beneficiary. The information includes description of the primary, secondary and tertiary extra corporeal causes of the principal health condition.

In yet another preferred embodiment of the present invention, the method further suggests, describes and/or demonstrates in an audio-visual-textual format, lifestyle modification tips to the beneficiary so as to increase the effectiveness of the aforementioned recommended healthcare products and to lower the health risk of principal health condition. The lifestyle modification tips include a primary tip, a secondary tip and a tertiary tip. The lifestyle modification tips are based on the principles of Ashtanga Yoga and Kriya Yoga. Ashtanga Yoga includes the practice of eight limbs of Yoga namely Yamas (behavioral observances), Niyamas (behavioral restrains), Asana (practice of physical postures), Pranayama (practice of breathing techniques), Pratyahara (withdrawal of senses), Dharana (concentration), Dhyana (meditation) and Samadhi (transcendence). Kriya yoga includes meditation techniques for accelerating an individual's spiritual growth. Inclusion of Ashtanga Yoga and Kriya Yoga based physical and metaphysical techniques for living life create a balance in all physiological processes. This balance then becomes adjuvant to any healthcare product's efficacy outcome. As a result, the health condition in context is managed in a more effective way.

In yet another aspect of the present invention, the method also provides the beneficiary with relevant and meritorious research articles about the principal health condition so as to educate him/her regarding the general aspects, scientific aspects and latest research advancements with respect to the principal health condition.

When all the above features are combined to yield the method of the present invention, as a result, the beneficiary is provided with an integrated approach towards managing the principal health condition thereby allowing him to manage the principal health condition in the most comprehensive and effective manner. By implementing a unique process for formulation of the healthcare products, by addressing corporeal component, extra corporeal component, associated health condition and the root cause factors of the principal health condition together, by suggesting lifestyle modifications along with increasing awareness of the beneficiary, the method of the present invention provides several advantages such as but not limited to increase in product potency and product efficiency, comprehensibility of end use, sustainability in terms of the effects and provision of value for purchase.

The overall functioning of the method of the present invention is described by means of an example as follows:

### Example

For explanatory purposes, an exemplary principal health condition—Alzheimer's disease is considered and the workflow of the method of the invention with this principal health condition in context, is as given in Table. 2.

TABLE 2

| Principal health condition | Alzheimer's disease |
|---|---|
| Associated health condition | Eye disorder |
| Root cause factor of principal | Liver function impairment in early |
| health condition | years of life |
| Principal healthcare product | Formulation containing Brahmi |
| | (Bacopa monierri leaf) |
| First ancillary healthcare | Formulation containing Clove |
| product | (Syzygium aromaticum) |
| Second ancillary healthcare | Formulation containing Haritaki |
| product | (Terminalia chebula) |
| Primary extra corporeal factor | Habit of Procrastination |
| Secondary extra corporeal factor | Overindulgence in doubts |
| Tertiary extra corporeal factor | Lack of self confidence |
| Primary lifestyle modification tip | Wake up before sun rise time at |
| | given geographical location |
| Secondary lifestyle modification tip | Abstinence from Alcohol |
| Tertiary lifestyle modification tip | Practicing Nadishuddhi Pranayam |

Referring to the example provided in Table. 2, the principal condition provided by the beneficiary is Alzheimer's disease. Physiologically, Alzheimer's disease is accompanied by some form of eye disorder, and hence eye disorder is considered as an associated condition with respect to Alzheimer's disease. Furthermore, the root cause factor for having Alzheimer's disease at a later stage in life is liver function impairment in early years of life. The method of the present invention provides the beneficiary with information regarding the association of eye disorder and liver function impairment at an early stage in life with Alzheimer's disease.

The method of the present invention further includes formulation of healthcare products to address the principal health condition, the associated health condition and the root cause factor of the principal health condition. In context of the present example, the principal healthcare product is formulated to address Alzheimer's disease directly and contains Brahmi (Bacopa monierri leaf). The first ancillary healthcare product is formulated to address the associated eye disorder and contains Clove (Syzygium aromaticum). The second ancillary healthcare product is formulated to address liver function impairment and contains Haritaki (Terminalia chebula). The method of the present invention further provides the beneficiary with a three product package comprising of the principal healthcare product, the first ancillary product and the second ancillary product.

The method of the present invention in yet another aspect provides the beneficiary with information regarding the extra corporeal factors related to the principal health condition. In context of the above example, where Alzheimer's disease is the principal condition, the first extra corporeal factor is the habit of procrastination, second extra corporeal factor is overindulgence in self-doubt, and the third extra corporeal factor is lack of self-confidence. In case of Alzheimer's disease, the first lifestyle modification tip suggested is waking up early, the second lifestyle modification tip suggested is abstinence from alcohol, and the third lifestyle modification tip suggested is the practice of Nadishuddhi Pranayam. These lifestyle modifications are based on the principles of Ashtanga Yoga and Kriya Yoga.

As far as the formulation of the above mentioned healthcare products based on the nine elements of life is concerned, an example of the ingredient Brahmi (Bacopa monierri leaf) to address the root cause factor of Alzheimer's disease in the second ancillary healthcare product is taken into consideration. In case of Alzheimer's, say for example, the proportion of the element Aakasha (symbolically space) in the formulation needs to be 50%. Naturally, Brahmi would have a specific composition of each of the five gross elements—Pruthvi (20%), Aakasha (20%), Jala (30%), Vayu (20%) and Tejas (20%). However, when used to treat and/or manage Alzheimer's disease, the proportion of the element Aakasha needs to be increased to 50% from 20%. This change in proportionality is brought about by means of specific processing and/or treatments of Brahmi.

As stated previously, the proportions of the five gross elements can be varied depending on the health condition while the proportions of the four subtle elements remain the same. It is to be noted that all the healthcare products contain all the nine elements. The proportions of these elements differ based on the principal health condition in context. It is to be further noted that the above mentioned values are only for explanatory purposes and may not correspond to the naturally occurring actual values.

In addition to all of the above, the method of the present invention also provides information to the beneficiary in an audio and/or video and/or textual format regarding the principal health condition and the lifestyle modifications.

While considerable emphasis has been placed herein on the specific elements of the preferred embodiment, it will be appreciated that many alterations can be made and that many modifications can be made in preferred embodiment without departing from the principles of the invention. These and other changes in the preferred embodiments of the invention will be apparent to those skilled in the art from the disclosure herein, whereby it is to be distinctly understood that the foregoing descriptive matter is to be interpreted merely as illustrative of the invention and not as a limitation.

What is claimed is:

1. A method for comprehensive management of a principal health condition, comprising the steps of;
receiving name of the principal health condition from a beneficiary, wherein the principal health condition has at least one corporeal component, at least one extra corporeal component, at least one associated health condition and at least one root cause factor;
explaining the principal health condition and the associated health condition to the beneficiary;
formulating plurality of healthcare products for the principal health condition, the associated health condition, and the root cause factors of the principal health condition, wherein the formulation of the healthcare products is based on nine elements of life;
recommending a package of three healthcare products, namely a principal healthcare product, a first ancillary healthcare product and a second ancillary healthcare product to the beneficiary, wherein the first principal healthcare product is formulated to manage the principal health condition, the first ancillary healthcare product is formulated to manage the associated health condition, and the second ancillary product is formulated to manage the root cause factors of the principal health condition;
providing information to the beneficiary regarding the extra corporeal components of the principal health condition;
providing the beneficiary with plurality of lifestyle modification tips based on the principles of Ashtanga Yoga and Kriya Yoga, so as to increase the effectiveness of the healthcare products and lower the risk of the principal health condition; and
increasing awareness of the beneficiary about the principal health condition by means of education, demonstration and provision of reference articles related to the principal health condition.

2. The method as claimed in claim 1, wherein the nine elements of life are Pruthvi, Jala, Tejas, Vayu, Akasha, Manas, Kaala, Disha, and Atman.

3. The method as claimed in claim 1, wherein the lifestyle modification tips include a primary tip, a secondary tip and a tertiary tip.

4. The method as claimed in claim 1, wherein the information regarding the extra corporeal component includes description of a primary factor, a secondary factor and a tertiary factor.

5. The method as claimed in claim 1, wherein the education and demonstration is in the form of but not limited to an audio, video or textual format.
